(19) United States
(12) Patent Application Publication
(10) Pub. No.: US 2026/0209190 A1
(43) Pub. Date: Jul. 23, 2026

(54) METHOD FOR PRODUCING ISOXAZOLINECARBOXYLIC ACID DERIVATIVES

(71) Applicant: Bayer Aktiengesellschaft, Leverkusen (DE)

(72) Inventors: Denise CIBU, Leverkusen (DE); Anton LISHCHYNSKYI, Leverkusen (DE)

(21) Appl. No.: 19/143,031

(22) PCT Filed: Dec. 20, 2023
(86) PCT No.: PCT/EP2023/087009

§ 371 Date: Jun. 25, 2025

(30) Foreign Application Priority Data
Dec. 28, 2022 (EP) 22216981.5

(51) Int. Cl. C07D 261/04; C07C 59/42; C07C 211/27; C07C 261/02; C07D 213/54

(52) U.S. Cl. CPC C07D 261/04; C07C 59/42; C07C 211/27; C07C 261/02; C07D 213/54

(57) ABSTRACT

The present invention relates to a novel process for preparing isoxazolinecarboxylic acid derivatives of the formula (I).

The present invention relates to a novel process for preparing isoxazolinecarboxylic acid derivatives of the formula (I).

Isoxazolinecarboxylic acid derivatives of the general formula (I) are important precursors for active agrochemical ingredients (for example for herbicides that are described in WO2014/048882 and WO2018/228985).

The prior art describes numerous cyclization methods for preparation of isoxazolinecarboxylic acid derivatives, for example Tetrahedron Letters, 1991, 6367-6370; Eur. J. Org. Chem. 2008, 5446-5460; Bull. Chem. Soc. Jpn. 1993, 2685. Possible transition states for the cycloaddition are discussed. Also disclosed are yields and isomer ratios depending on the reaction conditions. It was possible to achieve a high level of stereocontrol by chelate formation in the presence of appropriate metals, for example magnesium. However, a disadvantage in the processes described to date is the need to use dichloromethane (DCM) as solvent, which is not a preferred solvent for an industrial scale synthesis. Moreover, it was necessary to convert the allyl alcohols used as substrates in excess (2 equivalents). The use of DCM as solvent has likewise been described for the preparation of other isoxazoline derivatives, for example in the cyclization of aliphatic nitrile oxides with cyclic and acyclic allyl alcohols in the presence of 3.3 to 3.9 equivalents of 2-propanol and 3 equivalents of a Grignard reagent (for example by Carreira et al. Angew. Chem. Int. Ed. 2001, 40, 2082; J. Am. Chem. Soc. 2001, 123, 3611; Org. Lett. 2007, 9, 3857). If the compounds of the present invention are prepared by one of the methods known from the literature in solvents that are more advantageous for industrial scale use, for example tetrahydrofuran (THF), rather than DCM as solvent, this results in isomeric purities insufficient for an industrial scale synthesis.

It was thus an object of the invention to provide a process for preparing isoxazolinecarboxylic acid derivatives of the general formula (I), which is suitable for synthesis on an industrial scale and has a high yield and isomer purity, such that laborious purification methods can be dispensed with.

The object was achieved in accordance with the invention by a process for preparing isoxazolinecarboxylic acid derivatives of the general formula (I)

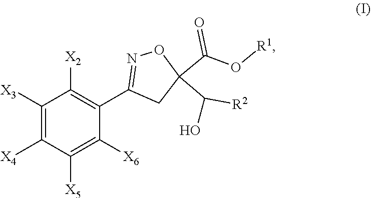

in which
X² is H, C1-C4-alkyl, C1-C4-fluoroalkyl, C1-C4-fluoroalkoxy, C1-C4-alkoxy, fluorine, CN,
X³ is H, C1-C4-alkyl, C1-C4-fluoroalkyl, C1-C4-fluoroalkoxy, C1-C4-alkoxy, fluorine, chlorine, CN,
X⁴ is H, C1-C4-alkyl, C1-C4-fluoroalkyl, C1-C4-fluoroalkoxy, C1-C4-alkoxy, fluorine, CN,
X⁵ is H, C1-C4-alkyl, C1-C4-fluoroalkyl, C1-C4-fluoroalkoxy, C1-C4-alkoxy, fluorine, chlorine, CN,
X⁶ is H, C1-C4-alkyl, C1-C4-fluoroalkyl, C1-C4-fluoroalkoxy, C1-C4-alkoxy, fluorine, CN,
R¹ is H, C1-C12-alkyl, unsubstituted benzyl or mono- or di-C1-C4-alkyl-substituted benzyl,
R² is C1-C4-alkyl,
characterized in that the compounds of the general formula (II)

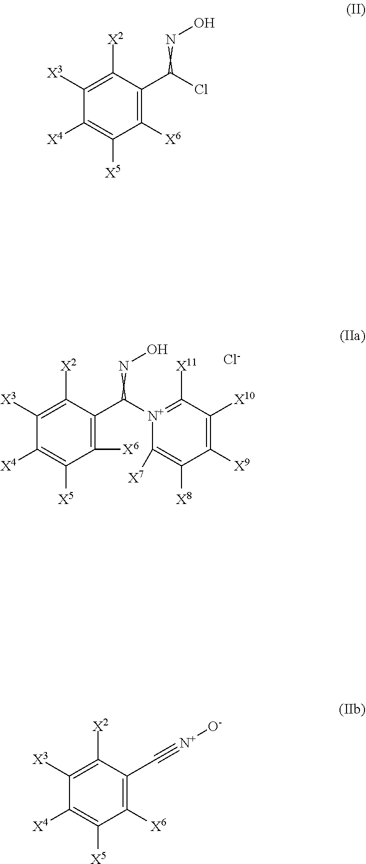

in which
X² to X⁶ have the definitions given above,
X⁷, X⁸, X¹⁰, X¹¹ are independently H or C₁-C₄-alkyl,
X⁹ are H, C₁-C₄-alkyl or N(C₁-C₄-alkyl)₂,
are reacted with compounds of the formula (III)

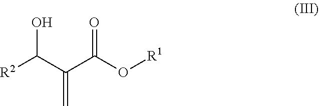

in which
R¹ and R² have the definitions given above—with the exception that R¹ is not H, with addition of a combination of reagents that enables formation of 1.0 to 2.0 equivalents of a reactive species “R³OMgHal” (IV)—based on compounds of the general formula (II),
R³ is alkyl, unsubstituted or alkyl-substituted benzyl and
Hal is halogen,
to give compounds of the general formula (I).

Preferred definitions of the radicals in the compounds of the general formulae (I), (II) and (III) are as follows:
X² is H, methyl, trifluoromethyl, difluoromethyl, difluoromethoxy, trifluoromethoxy, fluorine, methoxy, CN,
X³ is H, methyl, trifluoromethyl, difluoromethyl, difluoromethoxy, trifluoromethoxy, fluorine, chlorine, methoxy, CN,
X⁴ is H, methyl, trifluoromethyl, difluoromethyl, difluoromethoxy, trifluoromethoxy, fluorine, methoxy, CN,

X⁵ is H, methyl, trifluoromethyl, difluoromethyl, difluoromethoxy, trifluoromethoxy, fluorine, chlorine, methoxy, CN,
X⁶ is H, methyl, trifluoromethyl, difluoromethyl, difluoromethoxy, trifluoromethoxy, fluorine, methoxy, CN,
X⁷, X⁸, X⁹, X¹⁰, X¹¹ are independently H, methyl, ethyl,
X⁹ is H, methyl, ethyl or N(methyl)₂,
R¹ is H, C₁-C₄-alkyl,
R² is methyl, ethyl,
excluding R¹=H in the formula (III).

Preferably, the compounds of the general formula (IV) are generated by one of the following combinations of reagents:
R⁴MgHal and R³OH or
MgHal₂ and R³OM or
MgHal₂ and Mg(OR³)₂, where
R³ is C₂-C₁₂-alkyl, unsubstituted or C₁-C₄-alkyl-substituted benzyl and
Hal is halogen,
M is alkali metal,
R⁴ is alkyl, unsubstituted aryl, substituted aryl, unsubstituted benzyl, substituted benzyl, allyl, vinyl.

Alternatively, the compounds of the general formula (IV) are generated by the following combination of reagents:
R⁵MgHal and RR⁷CO where
R⁵ is C₁-C₆-alkyl, aryl, benzyl,
R⁶, R⁷ is H, C₁-C₆-alkyl, aryl,
and the resulting radical definition
R³ corresponds to R⁵R⁶R⁷C.

Preferred radical definitions for the reagents for preparation of compounds of the general formula (IV), presented by way of alternatives, are as follows:
R⁵ is C₁-C₄-alkyl, phenyl, benzyl, p-tolyl,
R⁶, R⁷ is H, C₁-C₄-alkyl, phenyl.

Particularly preferred definitions of the radicals in the compounds of the general formulae (I), (II) and (III) are as follows:
X² is H,
X³ is H, methyl, trifluoromethyl, difluoromethyl, fluorine, chlorine, methoxy, CN,
X⁴ is fluorine, H,
X⁵ is H, methyl, trifluoromethyl, difluoromethyl, fluorine, chlorine, methoxy, CN,
X⁶ is H,
X⁷, X⁸, X¹¹ are independently H, methyl, ethyl,
X⁹ is H, N(methyl)₂
X¹⁰ is H,
R¹ is H, methyl, ethyl, i-propyl, i-butyl,
R² is methyl,
excluding R¹=H in the formula (III).

More preferably the compounds of the general formula (IV) are generated by one of the following combinations of reagents:
R⁴MgHal and R³OH or
MgHal₂ and R³OM or
MgHal₂ and Mg(OR³)₂, where
R³ is C₂-C₈-alkyl,
Hal is bromine, chlorine,
M is alkali metal,
R⁴ is C₁-C₈-alkyl, phenyl, benzyl, p-tolyl, vinyl.

Very particularly preferred definitions of the radicals in the compounds of the general formulae (I), (II) and (III) are as follows:
X² is H,
X³ is H, fluorine,
X⁴ is H,
X⁵ is H, fluorine,
X⁶ is H,
X₈ is H, methyl, ethyl,
X⁷, X¹¹ is independently H, methyl, X⁹, X¹⁰
is H,
R¹ is H, methyl, i-propyl, i-butyl,
R² is methyl,
excluding R¹=H in the formula (III).

Even more preferably, the compounds of the general formula (IV) are generated by one of the following combinations of reagents:
R⁴MgHal and R³OH or
MgHal2 and R³OM or
MgHal2 and Mg(OR³)2, where
R³ is i-propyl, i-butyl, 2-butyl,
Hal is bromine, chlorine,
M is sodium,
R⁴ is methyl, ethyl, n-butyl, i-propyl.

Most preferred definitions of the radicals in the compounds of the general formulae (I), (II) and (III) are as follows:
X² is H,
X³ is fluorine,
X⁴ is H,
X⁵ is fluorine,
X⁶ is H,
X⁷, X⁸, X¹¹ is independently H, methyl,
X⁹, X¹⁰ is H,
R¹ is H, methyl, i-butyl,
R² is methyl,
excluding R¹=H in the formula (III).

### Definitions

Alkyl means saturated, straight-chain or branched hydrocarbyl radicals having the number of carbon atoms specified in each case, e.g. C₁-C₁₂-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Hal means halogen and is fluorine, chlorine, bromine or iodine. If the term is used for a radical, Hal means a fluorine, chlorine, bromine or iodine atom.

Alkali metal means lithium, sodium or potassium.

Aryl means phenyl or naphthyl.

The compounds of the formula (I) may take the form of isomer mixtures. The desired diastereomeric excess is increased by the optimized reaction conditions compared to the prior art. 1.7 to 2.0 equivalents of a reactive species “R³OMgHal” (IV) is especially advantageous.

When an (S)-alcohol is used, the main product is the (S,S)-diastereomer, and the (R,S)-diastereomer is the secondary diastereomer.

When an (R)-alcohol is used, the main product is the (R,R)-diastereomer, and the (S,R)-diastereomer is the secondary diastereomer. When a (rac)-alcohol is used, the main product is the (S,S/R,R)-mc-diastereomer and the (S,R/R,S)-rac-diastereomer is the secondary diastereomer.

The compounds of the formula (I) may be isolated as the corresponding ester or as the carboxylic acid after a hydrolytic workup.

The diastereomer ratio of up to 100:0 may be achieved in the compounds of the formula (I) via an enrichment by crystallization.

### Elucidation of the Processes and Intermediates

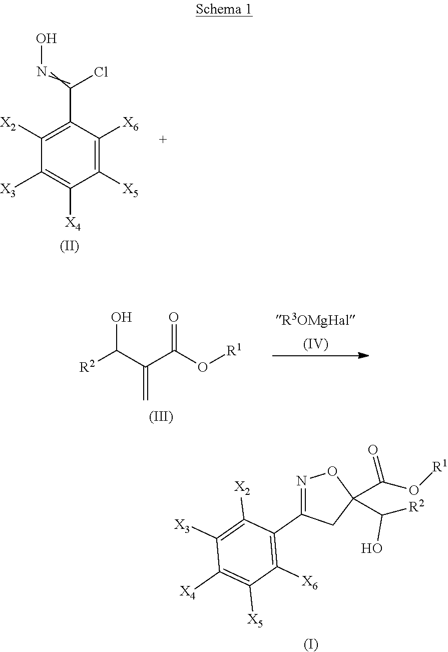

The object was achieved by a process for preparing isoxazolinecarboxylic acid derivatives of the formula (I), characterized in that the compounds of the general formula (II) are reacted with compounds of the formula (III) with addition of a combination of reagents that enables formation of 1.0 to 2.0 equivalents of a reactive species “R³OMgHal” (IV) based on compounds of the general formula (II) to give compounds of the general formula (I) (Scheme 1).

Preference is given to the use of 1.5 to 2.0 equivalents of the reactive species “R³OMgHal” (IV) based on compounds of the general formula (II).

Particular preference is given to the use of 1.7 to 2.0 equivalents of the reactive species “R³OMgHal” (IV) based on compounds of the general formula (II).

The addition of water may also be advantageous and may lead to a further improvement in the diastereomer ratio (d.r.). Preference is given to the use of up to 1.0 equivalent of water (based on compounds of the general formula (II)). Particular preference is given to the use of 0.1 up to 0.6 equivalent of water (based on compounds of the general formula (II)).

The cyclization is usually conducted within a temperature range from −25° C. to 70° C., preferably 10° C. to 30° C.

In addition, the cyclization is optionally conducted in the presence of a solvent or diluent or of a solvent mixture. The solvents are preferably toluene, xylene, tetrahydrofuran (THF), isopropyl acetate (i-PrOAc), acetonitrile, methyl tert-butyl ether (MTBE), methyl-THF, ethyl acetate (EtOAc) or mixtures in any ratios thereof.

The compounds of the general formula (III) are prepared via a two-stage process, and are known from the literature. The first stage is a Baylis-Hillman reaction. A relevant literature reference is: Drewes, S. E.; Hoole, R. F. A. [Synthetic Communications, 1985, vol. 15, 12, p. 1067-1074]. For the second stage, a relevant literature reference is: Nascimento et al. (2003, Tetrahedron Asymmetry 14, 311-311).

The compounds of the general formulae (II) and (III) are also known from WO 2018/228985. The preparation of the compounds of the formula (IIa) from (II) is known from Binenfeld, Zlatko; et al Glasnik Hemijskog Drustva Beograd (1966), 31(4-6), 243-50 and Daroszewski, J.; et al Pharmazie (1986), 41(10), 699-702.

### EXAMPLES

The present invention is elucidated in more detail by the examples which follow, without restricting the invention thereto (Table 1).

### Analysis Methods

The products were characterized by ¹H NMR spectroscopy and/or LC-MS (Liquid Chromatography Mass Spectrometry).

The NMR spectra were determined using a Bruker Avance 400 fitted with a flow probe head (volume 60 μl). In individual cases, the NMR spectra were measured with a Bruker Avance 11600. In quantitative NMR (qNMR) measurements, methyl 3,5-dinitrobenzoate was used as internal standard.

TABLE 1

| | i-PrOMgCl, | | Additional | qNMR | Diastereomer |
|---|---|---|---|---|---|
| Example ^{a)} | equivalents | | water/water | Yield ^{b)}, | ratio, d.r. |
| No. | (eq.) | R₁ | in (II), eq. | % | (NMR) |
| 1 | 2 | OMe | <0.05 | 83 | 88:12 |
| 2 ^{c)} | 2 | OMe | 0.3 | 86 | 92:8 |
| 3 ^{c)} | 1.9 | OMe | <0.05 | 86 | 87:13 |
| 4 ^{c)} | 1.9 | OMe | 0.3 | 86 | 91:9 |
| 5 ^{c)} | 1.8 | OMe | 0.3 | 87 | 89:11 |
| 6 ^{c)} | 1.7 | OMe | 0.3 | 86 | 85:15 |
| 7 | 2 | i-PrO | <0.05 | 85 | 93:7 |
| 8 | 2 | i-PrO | 0.3 | 89 | 93:7 |
| 9 | 1.9 | i-PrO | <0.05 | 85 | 91:9 |
| 10 | 1.9 | i-PrO | 0.3 | 88 | 92:8 |
| 11 | 1.8 | i-PrO | <0.05 | 86 | 92:8 |
| 12 | 1.8 | i-PrO | 0.3 | 88 | 92:8 |
| 13 | 1.7 | i-PrO | <0.05 | 83 | 89:11 |
| 14 | 1.7 | i-PrO | 0.3 | 86 | 89:11 |
| 15 | 2 | i-BuO | <0.05 | 87 | 94:6 |
| 16 | 2 | i-BuO | 0.3 | 91 | 95:5 |
| 17 | 1.9 | i-BuO | <0.05 | 81 | 94:6 |
| 18 | 1.9 | i-BuO | 0.3 | 88 | 95:5 |
| 19 | 1.8 | i-BuO | <0.05 | 85 | 93:7 |
| 20 | 1.8 | i-BuO | 0.3 | 90 | 94:6 |
| 21 ^{d)} | 1.8 | i-BuO | 0.3 | 81 (solids) | 98:2 |
| | | | | 8 (mother | 41:59 |
| | | | | liquor) | |
| 22 | 1.7 | i-BuO | <0.05 | 86 | 93:7 |
| 23 | 1.7 | i-BuO | 0.3 | 88 | 93:7 |
| 24 | 1.5 | i-BuO | <0.05 | 85 | 91:9 |
| 25 | 1.5 | i-BuO | 0.3 | 83 | 92:8 |
| 26 | 1.4 | i-BuO | <0.05 | 87 | 90:10 |
| 27 | 1.4 | i-BuO | 0.3 | 87 | 90:10 |
| 28 | 1.3 | i-BuO | <0.05 | 88 | 87:13 |
| 29 | 1.3 | i-BuO | 0.3 | 85 | 87:13 |

^{a)} Reactions were conducted with 3,5-difluoro-N-hydroxybenzenecarboximidoyl chloride.
^{b)} The yield of the product after hydrolysis with sodium hydroxide solution and acidification.
^{c)} (S)-Methyl 3-hydroxy-2-methylenebutanoate (0.524 g, 95.5% by weight) was used.
^{d)} Reactions were conducted with 0.524 mol of 3,5-difluoro-N-hydroxybenzenecarboximidoyl chloride. The corresponding carboxylic acid is isolated.

| | | | | |
|---|---|---|---|---|
| R¹, eq. | | | Yield, | Diastereomer |
| Reactant | Base, eq | Conditions | % | ratio, d.r. |

Prior art (X₂₋₆ = H)

| | | | | | |
|---|---|---|---|---|---|
| Bull. Chem. | Methyl, 2 | EtMgBr, 2 | CH₂Cl₂, −78 to −30° C./ | 100 | 92:8/89:11 |
| Soc. Jpn. | | | to R.T. | | |
| 1993, | Methyl, 1 | NEt₃, 1 | CH₂Cl₂, −30° C. | 90 | 30:70 |
| 2685-2689 | Methyl, 1 | EtMgBr, 1 | THF/CH₂Cl_{2,} | 93/86 | 63:37/81:19 |
| | | | −78 to −30° C. | | |

Conditions from the prior art, THF or THF/DCM as solvent (X_{2,4,6} = H, X_{3,5} = F)

| | | | | | |
|---|---|---|---|---|---|
| Example 30 | Methyl, 2 | 2-PrMgCl, 2 | THF, −78 to 15° C. | 88 | 81:19 |
| Example 31 | Methyl, 2 | 2-PrMgCl, 2 | THF/DCM, −78 to 15° C. | 87 | 85:15 |

### Example 1

Under an argon atmosphere, 2 equivalents (eq) of 2-propylmagnesium chloride (4.0 ml, 2 mol/l in THF) is initially charged at 20° C. 2 eq of 2-propanol (0.61 ml) is added dropwise over the course of 10 min while cooling (ice bath). Propane escapes (only slight foaming), and a white solid precipitates out. After the addition has ended and once the evolution of gas has ended, the resultant suspension is stirred at 20° C. for 20 minutes. Subsequently, 1 eq of methyl 3-hydroxy-2-methylenebutanoate (0.52 g, 99.8% by weight) is added dropwise at room temperature (RT). The mixture becomes more fluid. The suspension is stirred at RT for a further 15 min and then cooled to about 15° C. Subsequently, the solution of 3,5-difluoro-N-hydroxybenzenecarboximidoyl chloride in THF(2.55 g, 30.0%/aby weight) is added dropwise by means of a syringe pump at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. Toluene (1 ml) and an excess of aqueous sodium hydroxide solution are added to the residue, which is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (qNMR), 0.90 g of product is present in the residue (83%). Diastereomer ratio=88:12.

### Example 2

Under an argon atmosphere, 2 equivalents (eq) of 2-propylmagnesium chloride (4.23 ml, 1.87 mol/I in THF) is initially charged at 20° C. 2 eq of 2-propanol (0.61 ml) is added dropwise over the course of 10 min while cooling (ice bath). Propane escapes (only slight foaming), and a white solid precipitates out. After the addition has ended and once the evolution of gas has ended, the resultant suspension is stirred at 20° C. for 20 minutes. Subsequently, 1 eq of (S)-methyl 3-hydroxy-2-methylenebutanoate (0.545 g, 95.5% by weight) is added dropwise at room temperature (RT). The mixture becomes more fluid. The suspension is stirred at RT for a further 15 min and then cooled to about 15° C. Subsequently, the solution of 3,5-difluoro-N-hydroxybenzenecarboximidoyl chloride in a solvent mixture of toluene/THF (4.23 g, 18.1% by weight) is added dropwise by means of a syringe pump together with an additional 0.3 eq of water (22 μl) at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. Toluene (1 ml) and an excess of aqueous sodium hydroxide solution are added to the residue, which is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (qNMR), 0.93 g of product is present in the residue (86%). Diastereomer ratio=92:8.

### Example 13

Under an argon atmosphere, 1.7 equivalents (eq) of 2-propylmagnesium chloride (3.41 ml, 2 mol/I in THF) is initially charged at 20° C. 1.7 eq of 2-propanol (0.52 ml) is added dropwise over the course of 10 min while cooling (ice bath). Propane escapes (only slight foaming), and a white solid precipitates out. After the addition has ended and once the evolution of gas has ended, the resultant suspension is stirred at 20° C. for 20 minutes. Subsequently, 1 eq of 2-propyl 3-hydroxy-2-methylenebutanoate (0.79 g, 80.0% by weight) is added dropwise at room temperature (RT). The mixture becomes more fluid. The suspension is stirred at RT for a further 15 min and then cooled to about 15° C. Subsequently, the solution of 3,5-difluoro-N-hydroxybenzenecarboximidoyl chloride in THF (2.54 g, 30.1% by weight) is added dropwise by means of a syringe pump at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. Toluene (1 ml) and an excess of aqueous sodium hydroxide solution are added to the residue, which is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (qNMR), 0.90 g of product is present in the residue (83%). Diastereomer ratio=89:11.

### Example 14

Under an argon atmosphere, 1.7 equivalents (eq) of 2-propylmagnesium chloride (3.42 ml, 2 mol/I in THF) is initially charged at 20° C. 1.7 eq of 2-propanol (0.52 ml) is added dropwise over the course of 10 min while cooling (ice bath). Propane escapes (only slight foaming), and a white solid precipitates out. After the addition has ended and once the evolution of gas has ended, the resultant suspension is stirred at 20° C. for 20 minutes. Subsequently, 1 eq of 2-propyl 3-hydroxy-2-methylenebutanoate (0.79 g, 80.0% by weight) is added dropwise at room temperature (RT). The mixture becomes more fluid. The suspension is stirred at RT for a further 15 min and then cooled to about 15° C. Subsequently, the solution of 3,5-difluoro-N-hydroxybenzenecarboximidoyl chloride in a solvent mixture of toluene/THF (2.59 g, 29.7% by weight) is added dropwise by means of a syringe pump together with an additional 0.3 eq of water (22 μl) at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. Toluene (1 ml) and an excess of aqueous sodium hydroxide solution are added to the residue, which is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (qNMR), 0.93 g of product is present in the residue (86%). Diastereomer ratio=89:11.

### Example 21

Under an argon atmosphere, 1.8 equivalents (eq) of 2-propylmagnesium chloride (552 ml, 1.71 mol/I in THF) is initially charged at 20° C. 1.8 eq of 2-propanol (73 ml) is added dropwise over the course of 90 min while cooling (ice bath). The temperature was held between 15-30° C. Propane escapes (only slight foaming), and a white solid precipitates out. After the addition has ended and once the evolution of gas has ended, the resultant suspension is stirred for 40 minutes until an internal temperature of 20° C. has been attained. Subsequently, 1 eq of isobutyl 3-hydroxy-2-methylenebutanoate (93.90 g, 96.1% by weight) is added dropwise at a temperature between 20-25° C. over the course of 35 min. The mixture becomes more fluid. The suspension is stirred at RT for a further 75 min and then cooled to about 15° C. Subsequently, the solution of 3,5-difluoro-N-hydroxybenzenecarboximidoyl chloride in a solvent mixture of toluene/THF (233.0 g, 43.1% by weight) is added dropwise by means of a dropping funnel together with an additional 0.3 eq of water (2.83 ml) at about 15° C. (ice bath). The addition time is 1 hour (h) and 45 min. The internal temperature was kept at 15-20° C. After the addition has ended, the reaction mixture is warmed to RT and stirred for at least a further 2.5 h. Then the reaction mixture is added mixed with HCl solution (16.9% by weight, 140.4 g) while cooling with a water bath (20° C.), and the organic phase is removed. The water phase is subsequently washed with toluene, and the combined organic phases are concentrated under reduced pressure. Toluene (100 g), water (58 g) and an excess of aqueous sodium hydroxide solution (32 w %, 85.1 g) are added to the residue, which is stirred vigorously at 65° C. On completion of hydrolysis (2.5 h), the reaction mixture is cooled down to RT, and concentrated under reduced pressure to a thick suspension (60° C. bath, 240 mbar to 85 mbar). This is followed by acidification with sulfuric acid (20% by weight, 100 ml) while stirring vigorously. The product precipitates out, and is then filtered off and dried. In addition, 35 ml of 20% by weight sulfuric acid is added to the mother liquor (pH of mother liquor 1-2), and the mother liquor is extracted with isopropyl acetate (iPrOAc) (2×200 ml). The combined organic phases are concentrated under reduced pressure. The residue is combined with the solids and suspended in isopropyl acetate (iPrOAc) (500 ml) and stirred at 60° C. for 6 h. Subsequently, the suspension is concentrated to such an extent that it is still stirrable (50° C., 200 mbar to 150 mbar). 300 ml of toluene is added to the thick suspension. Distillation is continued at 50° C./150 mbar to 120 mbar until the suspension thickens again. The suspension is cooled down to room temperature and left to stand overnight until crystallization is complete. The suspension is then filtered and washed through with 290 ml of toluene, which leads to a solid product which is then dried. After determination of the content (qNMR) of the solid that is filtered off (119.20 g, 96.5% by weight), the product yield is 81%, diastereomeric ratio=98:2. There is 8% product in the concentrated mother liquor (43.13 g, 25.2% by weight). Diastereomeric ratio of the mother liquor corresponds to 41:59.

¹H NMR (401 MHz, DMSO-d₆): δ (ppm)=1.11 (d, J=6.5 Hz, 3H), 3.61 (d, J=17.8, 1H), 3.67 (d, J=17.8, 1H), 4.10 (q, J=6.4 Hz, 1H), 5.20 (bs, 1H), 7.34-7.45 (m, 3H), 13.28 (bs, 1H). ¹⁹F-NMR (376 MHz, DMSO-d₆): δ (ppm)=−108.7 (m, 2F).

### Example 22

Under an argon atmosphere, 1.7 equivalents (eq) of 2-propylmagnesium chloride (3.64 ml, 1.87 mol/l in THF) is initially charged at 20° C. 1.7 eq of 2-propanol (0.52 ml) is added dropwise over the course of 10 min while cooling (ice bath). Propane escapes (only slight foaming), and a white solid precipitates out. After the addition has ended and once the evolution of gas has ended, the resultant suspension is stirred at 20° C. for 20 minutes. Subsequently, 1 eq of isobutyl 3-hydroxy-2-methylenebutanoate (0.70 g, 98.2% by weight) is added dropwise at room temperature (RT). The mixture becomes more fluid. The suspension is stirred at RT for a further 15 min and then cooled to about 15° C. Subsequently, the solution of 3,5-difluoro-N-hydroxybenzenecarboximidoyl chloride in THF (2.54 g, 30.2% by weight) is added dropwise by means of a syringe pump at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. Toluene (1 ml) and an excess of aqueous sodium hydroxide solution are added to the residue, which is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (qNMR), 0.93 g of product is present in the residue (86%). Diastereomer ratio=93:7.

### Example 23

Under an argon atmosphere, 1.7 equivalents (eq) of 2-propylmagnesium chloride (3.87 ml, 1.76 mol/l in THF) is initially charged at 20° C. 1.7 eq of 2-propanol (0.52 ml) is added dropwise over the course of 10 min while cooling (ice bath). Propane escapes (only slight foaming), and a white solid precipitates out. After the addition has ended and once the evolution of gas has ended, the resultant suspension is stirred at 20° C. for 20 minutes. Subsequently, 1 eq of isobutyl 3-hydroxy-2-methylenebutanoate (0.70 g, 98.2% by weight) is added dropwise at room temperature (RT). The mixture becomes more fluid. The suspension is stirred at RT for a further 15 min and then cooled to about 15° C. Subsequently, the solution of 3,5-difluoro-N-hydroxybenzenecarboximidoyl chloride in a solvent mixture of toluene/THF (4.24 g, 18.1% by weight) is added dropwise by means of a syringe pump together with an additional 0.3 eq of water (22 μl) at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. Toluene (1 ml) and an excess of aqueous sodium hydroxide solution are added to the residue, which is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (qNMR), 0.95 g of product is present in the residue (88%). Diastereomer ratio=93:7.

### Example 30

2 ml of dry THF is initially charged under an argon atmosphere. Then 2 eq of (S)-methyl 3-hydroxy-2-methylenebutanoate (1.09 g, 95.5% by weight) is added at RT, and the solution is cooled down to −78° C. with a cooling bath (acetone/dry ice). Subsequently, 2 eq of 2-propylmagnesium chloride (4.55 ml, 1.76 mol/I in THF) is cautiously added dropwise at −78° C. while stirring. The reaction mixture is stirred at −78° C. for a further 40 min and then warmed up. The clear solution is then cooled to about 15° C. (water bath). Subsequently, the solution of 3,5-difluoro-N-hydroxybenzenecarboximidoyl chloride in THF (2.54 g, 30.1% by weight) is added dropwise by means of a syringe pump at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. Toluene (1 ml) and an excess of aqueous sodium hydroxide solution are added to the residue, which is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (qNMR), 0.95 g of product is present in the residue (88%). Diastereomer ratio=81:19.

TABLE 1

| | | | | | | Yield of |
|---|---|---|---|---|---|---|
| | | | | | Yield of | compound |
| | | | | | compound | of formula |
| | | | | | of formula | (IV)**, |
| | Base | Temperature | Reaction time | Conversion | (I), HPLC | HPLC |
| R¹ | (equivalents) | (° C.) | (h)* | (%) | area % | area % |
| isopropyl | N,N- | 155-160 | 20.5 | 98 | 85 | 86 |
| | dimethylcyclo- | | | | | |
| | hexylamine | | | | | |
| | (3) | | | | | |
| isopropyl | N,N- | 155-160 | 20.5 | 98 | 87 | 84 |
| | dimethylcyclo- | | | | | |
| | hexylamine | | | | | |
| | (5) | | | | | |
| 2- | N,N- | 160 | 17 | 99 | 86 | not |
| methylpropan- | dimethylcyclo- | | | | | determined |
| 1-yl | hexylamine | | | | | |
| | (5) | | | | | |
| n-butyl | N,N- | 160 | 14 | 98 | 83 | not |
| | dimethylcyclo- | | | | | determined |
| | hexylamine | | | | | |
| | (5) | | | | | |
| cyclohexyl | N,N- | 155 | 19 | 99 | 90 | not |
| | dimethylcyclo- | | | | | determined |
| | hexylamine | | | | | |
| | (5) | | | | | |
| propyl | N,N- | 152 | 13 | 98 | 75 | not |
| | dimethylcyclo- | | | | | determined |
| | hexylamine | | | | | |
| | (5) | | | | | |
| ethyl | N,N- | 160 | 7 | 98 | 58 | not |
| (comparative) | dimethylcyclo- | | | | | determined |
| | hexylamine | | | | | |
| | (5) | | | | | |
| methyl | N,N- | 130-138 | 1.33 | 99 | 1.7 | not |
| (comparative) | dimethylcyclo- | | | | | determined |
| | hexylamine | | | | | |
| | (5) | | | | | |

*The reaction was ended when virtually complete conversion of the reactant compound of the formula (II) was observed by HPLC.
**after hydrolysis according to step 2 described by way of example above
It is apparent from Table 1 that the resultant yield of compounds of the formula (I) is highly dependent on the selection of variable R¹.

1. A process for preparing isoxazolinecarboxylic acid derivatives of the formula (I) 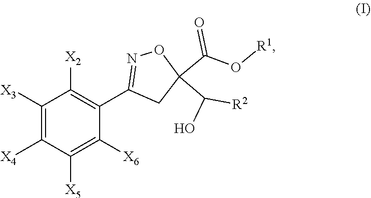
wherein,
X² is H, C1-C4-alkyl, C1-C4-fluoroalkyl, C1-C4-fluoroalkoxy, C1-C4-alkoxy, fluorine, CN;
X³ is H, C1-C4-alkyl, C1-C4-fluoroalkyl, C1-C4-fluoroalkoxy, C1-C4-alkoxy, fluorine, chlorine, CN;
X⁴ is H, C1-C4-alkyl, C1-C4-fluoroalkyl, C1-C4-fluoroalkoxy, C1-C4-alkoxy, fluorine, CN;
X⁵ is H, C1-C4-alkyl, C1-C4-fluoroalkyl, C1-C4-fluoroalkoxy, C1-C4-alkoxy, fluorine, chlorine, CN;
X⁶ is H, C1-C4-alkyl, C1-C4-fluoroalkyl, C1-C4-fluoroalkoxy, C1-C4-alkoxy, fluorine, CN;
R¹ is H, C1-C12-alkyl, unsubstituted benzyl or mono- or di-C1-C4-alkyl-substituted benzyl;
R² is C1-C4-alkyl;
characterized in that the compounds of the general formula (II) 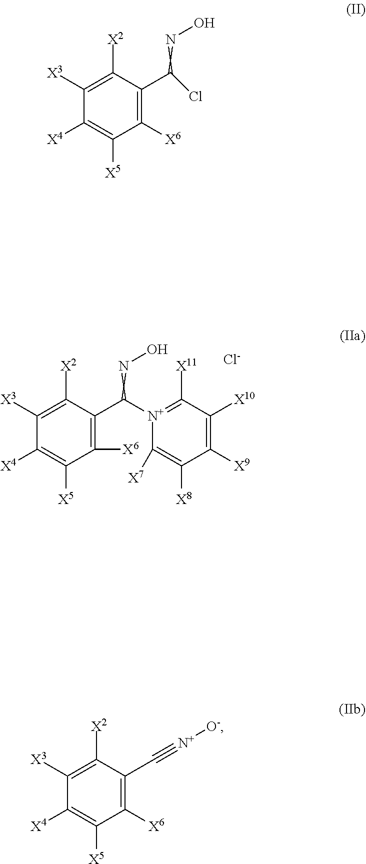
wherein,
X² to X⁶ have the definitions given above;
X⁷, X⁸, X¹⁰, X¹¹ are independently H or C1-C4-alkyl; and
X⁹ are H, C1-C4-alkyl or N(C1-C4-alkyl)2;
are reacted with compounds of the formula (III) 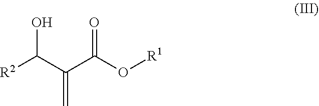
wherein,
R¹ and R² have the definitions given above with the exception that R¹ is not H, with addition of a combination of reagents that enables formation of 1.0 to 2.0 equivalents of a reactive species “R³OMgHal” (IV) based on compounds of the general formula (II),
wherein
R³ is alkyl, unsubstituted or alkyl-substituted benzyl; and
Hal is halogen wherein,
to give compounds of the general formula (I).

2. The process according to claim 1, wherein the definitions of the radicals in the compounds of the general formulae (I), (II) and (III) are as follows:
X² is H, methyl, trifluoromethyl, difluoromethyl, difluoromethoxy, trifluoromethoxy, fluorine, methoxy, CN;
X³ is H, methyl, trifluoromethyl, difluoromethyl, difluoromethoxy, trifluoromethoxy, fluorine, chlorine, methoxy, CN;
X⁴ is H, methyl, trifluoromethyl, difluoromethyl, difluoromethoxy, trifluoromethoxy, fluorine, methoxy, CN;
X⁵ is H, methyl, trifluoromethyl, difluoromethyl, difluoromethoxy, trifluoromethoxy, fluorine, chlorine, methoxy, CN;
X⁶ is H, methyl, trifluoromethyl, difluoromethyl, difluoromethoxy, trifluoromethoxy, fluorine, methoxy, CN;
X⁷, X⁸, X¹⁰, X¹¹ are independently H, methyl, ethyl;
X⁹ is H, methyl, ethyl or N(methyl)2;
R¹ is H, C1-C4-alkyl;
R² is methyl, ethyl; and
excluding R¹=H in the formula (III).

3. The process according to claim 1, wherein the definitions of the radicals in the compounds of the general formulae (I), (II) and (Ill) are as follows:
X² is H;
X³ is H, methyl, trifluoromethyl, difluoromethyl, fluorine, chlorine, methoxy, CN;
X⁴ is fluorine, H;
X⁵ is H, methyl, trifluoromethyl, difluoromethyl, fluorine, chlorine, methoxy, CN;
X⁶ is H;
X⁷, X⁸, X¹¹ are independently H, methyl, ethyl;
X⁹ is H, N(methyl)2;
X¹⁰ is H;
R¹ is H, methyl, ethyl, i-propyl, i-butyl;
R² is methyl; and
excluding R¹=H in the formula (III).

4. The process according to claim 1, wherein the definitions of the radicals in the compounds of the general formulae (I), (II) and (III) are as follows:
X² is H;
X³ is H, fluorine;
X⁴ is H;
X⁵ is H, fluorine;
X⁶ is H;
X⁸ is H, methyl, ethyl;
X⁷, X¹¹ is independently H, methyl;
X⁹, X¹⁰ is H;
R¹ is H, methyl, i-propyl, i-butyl;
R² is methyl; and
excluding R¹=H in the formula (III).

5. The process according to claim 1, wherein the definitions of the radicals in the compounds of the general formulae (I), (II) and (III) are as follows:
X² is H;
X³ is fluorine;
X⁴ is H;
X⁵ is fluorine;
X⁶ is H;
X⁷, X⁸, X¹¹ is independently H, methyl;
X⁹, X¹⁰ is H;
R¹ is H, methyl, i-butyl;
R² is methyl; and
excluding R¹=H in the formula (III).

6. The process according to claim 1, wherein the definitions of the radicals in the compounds of the general formula (IIa) are as follows:
X⁷, X⁸, X¹⁰, X¹¹ are independently H, methyl, ethyl; and
X⁹ is H, methyl, ethyl or N(methyl)2.

7. The process according to claim 1, wherein the definitions of the radicals in the compounds of the general formula (IIa) are as follows:
X⁷, X¹¹ is H;
X⁸, X¹⁰ are independently H, methyl, ethyl; and
X⁹ is H, methyl, ethyl, N(methyl)₂.

8. The process according to claim 1, wherein the compound of the general formula (IV) is generated by one of the following combinations of reagents:
R⁴MgHal and R³OH or
MgHal₂ and R³OM or
MgHal₂ and Mg(OR³)₂, wherein,
R³ is C₂-C₁₂-alkyl, unsubstituted or C₁-C₄-alkyl-substituted benzyl;
Hal is halogen;
M is alkali metal; and
R⁴ is alkyl, unsubstituted aryl, substituted aryl, unsubstituted benzyl, substituted benzyl, allyl, vinyl.

9. The process according to claim 1, wherein the compound of the general formula (IV) is generated by one of the following combinations of reagents:
R⁴MgHal and R³OH or
MgHal2 and R³OM or
MgHal2 and Mg(OR³)₂, wherein,
R³ is C2-C8-alkyl;
Hal is bromine or chlorine;
M is alkali metal; and
R⁴ is C1-C8-alkyl, phenyl, benzyl, p-tolyl, vinyl.

10. The process according to claim 1, wherein the compound of the general formula (IV) is generated by one of the following combinations of reagents:
R⁴MgHal and R³OH or
MgHal2 and R³OM or
MgHal2 and Mg(OR³)₂, wherein,
R³ is C2-C8-alkyl;
Hal is bromine or chlorine;
M is alkali metal; and
R⁴ is C1-C4-alkyl, phenyl, benzyl, p-tolyl, vinyl.

11. The process according to claim 1, characterized in that 1.3 to 2.0 equivalents of the reactive species “R³OMgHal” (IV) is used, based on compounds of the general formula (II).

12. The process according to claim 1, characterized in that 1.7 to 2.0 equivalents of the reactive species “R³OMgHal” (IV) is used, based on compounds of the general formula (II).

13. The process according to claim 1, characterized in that 0.1 to 0.6 equivalent of additional water is used, based on the formula (II).

14. The process according to claim 1, characterized in that the solvent is toluene, xylene, tetrahydrofuran (THF), isopropyl acetate (i-PrOAc), acetonitrile, methyl tert-butyl ether (MTBE), methyl-THF, ethyl acetate (EtOAc) or mixtures in any ratios thereof.

15. The process according to claim 1, characterized in that the reaction is conducted at −25° C. to 70° C.

16. The process according to claim 1, characterized in that the reaction is conducted at 10° C. to 30° C.

17. The process according to claim 1, characterized in that the diastereomeric ratio is increased by a further crystallization step.

18. The process according to claim 1, characterized in that the compounds of the general formula (IV) are generated via a Grignard reaction, with the following combinations of reagents:
R⁵MgHal and R⁶R⁷CO wherein
R⁵ is C1-C6-alkyl, aryl, benzyl; and
R⁶, R⁷ are H, C1-C6-alkyl, aryl, and the resulting radical definition corresponds to R³R⁵R⁶R⁷C.
